(19) United States
(12) Patent Application Publication
(10) Pub. No.: US 2013/0302864 A1
(43) Pub. Date: Nov. 14, 2013

(54) Method for Producing a Fatty Acid Ester

(71) Applicant: AJINOMOTO CO., INC., Tokyo (JP)

(72) Inventor: Shigeo Suzuki, Kanagawa (JP)

(21) Appl. No.: 13/944,197

(22) Filed: Jul. 17, 2013

Related U.S. Application Data
(63) Continuation of application No. PCT/JP2012/050975, filed on Jan. 18, 2012.

(30) Foreign Application Priority Data
Jan. 18, 2011 (JP) 2011-008272

(51) Int. Cl. C12P 7/64

(52) U.S. Cl. CPC C12P 7/6436
USPC 435/134

(57) ABSTRACT

A method for producing a fatty acid ester, which comprises: (a) incubating a culture of microalga at a mid-temperature, wherein the culture is obtained by culturing the microalga in a medium, (b) then adding an alcohol, allowing a reaction at a temperature lower than the mid-temperature, and (c) collecting a fatty acid ester from an obtained reaction product.

This application is a Continuation of, and claims priority under 35 U.S.C. §120 to, International Application No. PCT/JP2012/050975, filed Jan. 18, 2012, and claims priority therethrough under 35 U.S.C. §119 to Japanese Patent Application No. 2011-008272, filed Jan. 18, 2011, the entireties of which are incorporated by reference herein.

### BACKGROUND OF THE INVENTION

1. Field of the Invention

The present invention relates to a method for producing a fatty acid ester using algae. Fatty acid esters are used in various fields, such as those of food additives, chemicals, cosmetics, and drugs.

2. Brief Description of the Related Art

Fatty acid esters are industrially produced from fats and oils derived from animals, plants, fishes, and waste oils by the transesterification method. Known methods of transesterification include using a catalyst of an acid, an alkali, a metal, a lipase, or the like. Examples include, for example, the methods disclosed in U. Schuchardt et al., 1998, J. Brazilian Chemical Society, 9 (3), 199-210, H. Fukuda, A. Kondo, H. Noda, 2001, J. Bioeng, 92, 405-416, T. Samukawa et al., 2000, J. Biosci. and Bioeng., 90, 180-183, and L. A. Nelson et al., 1996, J. Am. Oil Soc. Chem., 73, 1191-1195. Other than the methods utilizing a catalyst, the supercritical method can be used. Examples include, for example, the methods disclosed in S. Saka, D. Kusdiana, 2001, Fuel, 80, 225-231, D. Kusdiana, S. Saka, 2001, J. Chem. Eng. Japan, 34, 383-387, and D. Kusdiana, S. Saka, 2001, Fuel, 80, 693-698.

In the industrial production of fatty acid esters based on transesterification, fish oils, animal oils, vegetable oils, waste oils, and so forth can be used as fats and oils. Fats and oils derived from higher plants, such as soybean and palm, are frequently used as a source of fat or oil in the method for producing a fatty acid ester by transesterification. These are fats and oils easily industrially obtainable from seeds by compression or solvent extraction. On the other hand, fats and oils contained in microalgae are present at amount comparable to that of soybean or palm seeds in terms of dry weight, but dry alga body weight in culture of microalgae is less than 1% of the culture medium. In addition, the process of separating alga bodies, dehydrating them, disrupting cells, extracting fats and oils, and purifying them is complicated and difficult. It is possible to produce a fatty acid ester from fat or oil purified from algae by using an acid, an alkali, or a lipase (International Patent Publication WO2010/000416, N. Nagle, P. Lemke, 1990, Applied Biochem. and Biotech., 24, 355-361, and A. Robles Medina et al., 1999, J. Biotech., 70, 379-391). Further, in the methods of U.S. Patent Published Application No. 20080241902, China Patent Published Application No. 101580857, and U.S. Patent Published Application No. No. 20090158638, an alcohol is added to a microalga, and transesterification of fats and oils are directly induced within cells, but all the methods require an acid or an alkaline catalyst for the transesterification.

It is known that Synechocystis algae, which are typical recombinant producible algae, can produce a large amount of fatty acids, since they express acetyl-CoA carboxylase and thioesterase (X. Liu et al., 2009, PNAS, 24, 1-6), and they can produce triglycerides, since they express diacylglycerol acetyltransferase (U.S. Patent Published Application No. No. 20100081178). Therefore, it is easy to produce a fatty acid ester from fats and oils of Synechocystis algae by using a catalyst of an acid, an alkali, a lipase or the like. Furthermore, it is known that Synechocystis algae can be made to express pyruvate decarboxylase and alcohol dehydrogenase, and thereby made to produce ethanol, and a fatty acid ester can be produced within the cells with ethanol acetyltransferase (International Patent Publication WO2010/011754). However, methods for producing a fatty acid ester from fat or oil within alga cells without using genetic recombination techniques have not been previously reported.

Algae generally use lipases for decomposition of lipids of cell membranes or fats and oils (K. Hoehne-Reitan et al., 2007, Aqua. Nutri., 13, 45-49). Furthermore, increase in the lipase activity and decomposition of fats and oils into fatty acids induced by silica starvation have been confirmed in diatoms (N. Nagle et al., 1989, Energy from Biomass and Wastes, 12, 1107-1115), but production of a fatty acid ester in alga cells by adding an alcohol to such fatty acids has not been previously reported.

Furthermore, it is known to extract organic substances from Chlorella algae by subjecting them to a high temperature treatment to disrupt cells thereof (Japanese Patent Laid-open (KOKAI) No. 9-75094, International Patent Publication W02006/095964 and U.S. Patent Published Application No. 20070202582), but direct conversion of fats and oils in the cells into fatty acid esters has not been reported. Furthermore, although it is also known that autolysis of Chlorella algae at 40 to 55° C. increases low molecular weight nucleic acid-related compounds (Japanese Patent Laid-open (KOKAI) No. 62-278977), production of fatty acid esters using this process has not been reported so far.

### SUMMARY OF THE INVENTION

It is one of numerous aspects of the present invention to provide a more efficient method for producing a fatty acid ester, especially a lower cost fatty acid ester-producing method not requiring the addition of a catalyst, compared with conventional fatty acid ester-producing methods utilizing fats and oils derived from animals, plants, fishes and waste fluids as substrates and utilizing an acid or alkaline catalyst.

It was found that by reacting a culture of an alga at a mid-temperature before a reaction with an alcohol, fatty acid esters could be efficiently produced in algae cells without adding an acid or an alkali. The present invention thus provides the following:

It is an aspect of the present invention to provide a method for producing a fatty acid ester, which comprises:
(a) incubating a culture of a microalga at a mid-temperature,
(b) adding an alcohol to the culture, and reducing the temperature of the culture to a temperature lower than the mid-temperature, and
(c) collecting a fatty acid ester from an obtained reaction product. It is an aspect of the present invention to provide the method as described above, wherein said mid-temperature is 40° C. or higher.

It is an aspect of the present invention to provide the method as described above, wherein said mid-temperature is 70° C. or lower.

It is an aspect of the present invention to provide the method as described above, wherein said incubating of (a) is performed at a weakly acidic to weakly alkaline pH.

It is an aspect of the present invention to provide the method as described above, wherein said temperature lower than the mid-temperature is 5° C. or higher.

It is an aspect of the present invention to provide the method as described above, wherein said temperature lower than the mid-temperature is 60° C. or lower.

It is an aspect of the present invention to provide the method as described above, wherein said alcohol concentration is 5% or higher.

It is an aspect of the present invention to provide the method as described above, wherein said alcohol concentration is 70% or lower.

It is an aspect of the present invention to provide the method as described above, wherein said alcohol is a lower alcohol having a carbon number of 5 or smaller.

It is an aspect of the present invention to provide the method as described above, wherein said alcohol is a higher alcohol having a carbon number of 6 or larger.

It is an aspect of the present invention to provide the method as described above, wherein a product obtained after step (b) is treated with an organic solvent to extract a fatty acid ester, and the fatty acid ester is collected from an obtained extract.

It is an aspect of the present invention to provide the method as described above, wherein a product obtained after step (b) is subjected to centrifugation, followed by treating the resulting precipitate with an organic solvent.

It is an aspect of the present invention to provide the method as described above, wherein the organic solvent is selected from the group consisting of methanol, ethanol, 2-propanol, acetone, butanol, pentanol, hexanol, heptanol, octanol, chloroform, methyl acetate, ethyl acetate, dimethyl ether, diethyl ether, and hexane.

It is an aspect of the present invention to provide the method as described above, wherein the microalga is an alga belonging to the phylum Chlorophyta.

It is an aspect of the present invention to provide the method as described above, wherein the microalga is an alga belonging to the class Chlorophyceae, Trebouxiophyceae, or Prasinophyceae.

It is an aspect of the present invention to provide the method as described above, wherein the microalga is an alga belonging to the class Chlorophyceae.

Fatty acid esters can be efficiently produced by using the method as described herein.

### BRIEF DESCRIPTION OF THE DRAWINGS

FIG. 1 shows results of examination of temperature conditions for the reaction of the first step in the two-step reaction of alga culture.

FIG. 2 shows results of examination of time for the reaction of the first step in the two-step reaction of alga culture.

FIG. 3 shows results of examination of pH for the reaction of the first step in the two-step reaction of alga culture.

FIGS. 4A and B show results of examination of methanol addition concentration for the reaction of the second step in the two-step reaction of alga culture.

FIGS. 5A and B show results of examination of time for the reaction of the second step in the two-step reaction of alga culture.

FIGS. 6A and B show results of examination of temperature for the reaction of the second step in the two-step reaction of alga culture.

FIG. 7 shows results of examination of type of alcohol added in the reaction of the second step in the two-step reaction of alga culture.

FIG. 8 shows results of identification of fatty acid alcohol esters produced by the two-step reaction of alga culture.

FIG. 9 shows a result of two-step reaction performed by using the Scenedesmus abundans UTEX 1358 strain.

### DESCRIPTION OF THE PREFERRED EMBODIMENTS

Hereafter, the present invention will be explained in detail.

<1>Microalgae and Culture Method Therefor

As the microalga, any algae can be used. However, microalgae which accumulate starches and/or fats and oils in alga bodies are particular examples.

Algae can refer to all organisms performing oxygen generating type photosynthesis except for Bryophyta, Pteridophyta and Spermatophyta, which live mainly on the ground. Algae can include various unicellular organisms and multicellular organisms such as cyanobacteria (blue-green algae), which are prokaryotes, as well as those classified into the phyla Glaucophyta, Rhodophyta (red algae), Chlorophyta, Cryptophyta (crypt algae), Haptophyta (haptophytes), Heterokontophyta, Dinophyta (dinoflagellates), Euglenophyta, or Chlorarachniophyta, which are eukaryotes. Microalgae can refer to algae having a microscopic structure among these algae except for marine algae, which are multicellular organisms (Biodiversity Series (3) Diversity and Pedigree of Algae, edited by Mitsuo Chihara, Shokabo Publishing Co., Ltd. (1999)).

It is known that microalgae include those accumulating fats and oils as storage substances (Chisti Y., 2007, Biotechnol. Adv., 25:294-306). As such algae, those belonging to the phylum Chlorophyta or Heterokontophyta are well known. Examples of the algae belonging to the phylum Chlorophyta include those belonging to the class Chlorophyceae, and examples of algae belonging to the class Chlorophyceae include Chlorella minutissima (Bhatnagar A., 2010, Appl. Biochem. Biotechnol., 161:523-36), Scenedesmus obliquus (Shovon, M. et al., 2009, Appl. Microbiol. Biotechnol., 84:281-91), Neochloris oleoabundans (Tornabene, T. G. et al., 1983, Enzyme and Microb. Technol., 5:435-440), Nannochloris sp. (Takagi, M. et al., 2000, Appl. Microbiol. Biotechnol., 54:112-117) and so forth. In the phylum Heterokontophyta, the classes Chrysophyceae, Dictyochophyceae, Pelagophyceae, Rhaphidophyceae, Bacillariophyceae, Phaeophyceae, Xanthophyceae, and Eustigmatophyceae are classified. Examples of frequently used algae belonging to the class Bacillariophyceae include Thalassiosira pseudonana (Tonon, T. et al., 2002, Phytochemistry, 61:15-24). Specific examples of Chlorella minutissima include the Chlorella minutissima UTEX 2314 strain, specific examples of Scenedesmus obliquus include the Scenedesmus obliquus UTEX393 strain, specific examples of Neochloris oleoabundans include the Neochloris oleoabundans UTEX 1185 strain, specific examples of Nannochloris sp. include the Nannochloris sp. UTEX LB 1999 strain, and specific examples of Thalassiosira pseudonana include the Thalassiosira pseudonana UTEX LB FD2 strain. These strains can be obtained from the University of Texas at Austin, The Culture Collection of Algae (UTEX), 1 University Station A6700, Austin, Tex. 78712-0183, USA.

Further, as algae that produce EPA and DHA, which are highly functional fatty acids, those belonging to the phylum Chlorophyta, Heterokontophyta, Rhodophyta, or Haptophyta are known well. Examples of algae belonging to the phylum Chlorophyta include algae belonging to the class Chlorophyceae, Prasinophyceae, or Trebouxiophyceae, and examples of well-known algae belonging to the class Chlorophyceae include Chlorella minutissima (Rema V. et al., 1998, JAOCS. 75:393-397). Examples of algae belonging to the phylum Heterokontophyta include algae belonging to the class Bacillariophyceae or Eustigmatophyceae, examples of algae belonging to the class Bacillariophyceae and frequently used include Thalassiosira pseudonana (Tonon, T. et al., 2002, Phytochemistry 61:15-24), and examples of algae belonging to the class Eustigmatophyceae include Nannochloropsis oculata.

There is much information about culture of microalgae, and those of the genus Chlorella or Arthrospira (Spirulina), Dunaliella salina and so forth are industrially cultured on a large scale for use as food (Spolaore, P. et al., 2006, J. Biosci. Bioeng., 101:87-96). For Chlamydomonas reinhardtii, for example, the 0.3× HSM medium (Oyama Y. et al., 2006, Planta, 224:646-654) can be used, and for Chlorella kessleri, the 0.2× Gamborg's medium (Izumo A. et al., 2007, Plant Science, 172:1138-1147) and so forth can be used. For Chlorella vulgaris, the BG-11 medium, the M8 medium (Ramkumar, K. M. et al., 1998, Biotech. Bioeng., 59:605-611) and so forth can be used. Neochloris oleoabundans and Nannochloris sp. can be cultured by using the modified NORO medium (Yamaberi, K. et al., 1998, J. Mar. Biotechnol., 6:44-48; Takagi, M. et al., 2000, Appl. Microbiol. Biotechnol., 54:112-117), the Bold's basal medium (Tornabene, T. G. et al., 1983, Enzyme and Microb. Technol., 5:435-440; Archibald, P. A. and Bold, H. C., 1970, Phytomorphology, 20:383-389), or the Daigo's IMK medium (Ota M. et al., 2009, Bioresource Technology, 100:5237-5242). For Thalassiosira pseudonana as an alga belonging to the class Bacillariophyceae, the F/2 medium (Lie, C.-P. and Lin, L.-P., 2001, Bot. Bull. Acad. Sin., 42:207-214) and so forth can be used. Further, a photobioreactor can also be used for culture of microalgae (WO2003/094598).

The culture can be performed by adding 1 to 50% of preculture fluid based on the volume of main culture in many cases. Initial pH can be around neutral, i.e., 7 to 9, and the pH does not need to be adjusted during culture in many cases. However, the pH may be adjusted if needed. The culture temperature can be 25 to 35° C., and in particular, a temperature of around 28° C. is generally frequently used. However, the culture temperature may be a temperature suitable for the alga to be used. Air is blown into the culture medium in many cases, and as aeration rate, an aeration volume per unit volume of culture medium per minute of 0.1 to 2 vvm (volume per volume per minute) can be frequently used. Further, CO₂ may also be blown into the medium in order to promote growth, and it can be blown at about 0.5 to 5% of the aeration rate. Although optimum illumination intensity of light also differs depending on type of microalgae, an illumination intensity of about 1,000 to 30,000 luxes can be frequently used. As the light source, it is common to use a white fluorescent lamp indoors, but the light source does not have to be so limited. It is also possible to perform the culture outdoors with sunlight. The culture medium may be stirred at an appropriate intensity, or circulated, if needed. Further, it is known that algae accumulate fats and oils in alga bodies when nitrogen source is depleted (Thompson G. A. Jr., 1996, Biochim. Biophys. Acta, 1302:17-45), and a medium of a limited nitrogen source concentration can also be used for the main culture.

The culture of microalga can include a culture fluid containing alga bodies and alga bodies collected from a culture fluid.

Alga bodies can be collected from a culture fluid by usual centrifugation, filtration, gravitational precipitation using a flocculant, or the like (Grima, E. M. et al., 2003, Biotechnol. Advances, 20:491-515).

It is one example to concentrate the microalga by centrifugation or the like, before the reaction of them at a mid-temperature. The concentration operation of alga bodies can include removing solvent component to obtain a concentration of 25 g/L or higher, or 250 g/L or higher, as a concentration of microalga in terms of dry weight in the solution (including suspending alga bodies separated from a medium by centrifugation or the like in a liquid to obtain a desired concentration), and a process of precipitating alga bodies, separating them from a medium and using them.

<2>Method for Reaction of Culture of Microalga and Reaction Product

The two-step reaction of culture of a microalga with a reaction at a mid-temperature and a reaction at a sub-mid-temperature (temperature lower than the mid-temperature) can be performed (i.e., such two-step reaction is induced), and the product (reaction product) can be used for collecting a fatty acid ester.

The reaction product of a microalga can mean a reaction mixture in which the two-step reaction of culture of the microalga with a reaction at a mid-temperature and a reaction at a sub-mid-temperature after addition of an alcohol has been allowed. The reaction mixture which has undergone the two-step reaction may be further subjected to extraction, fractionation, and/or another treatment, so long as the subsequent collection of a fatty acid ester is not inhibited. In the reaction product, by-products are produced in addition to the fatty acid ester, and among them, glycerol generated by the transesterification of fats and oils may be used for production of L-amino acids using bacteria able to produce L-amino acid(s) or other chemical products.

The reaction of the second step of the two-step reaction can be a reaction that generates a fatty acid ester. The reaction of the first step can be a reaction which alters the state of the culture of the microalga so that the fatty acid ester generation reaction of the second step is promoted.

The temperatures of the two-step reaction may be temperatures that are sufficient for increasing the fatty acid ester in the reaction product obtained after the reaction at a mid-temperature, addition of an alcohol, and the reaction at a sub-mid-temperature. After the reaction of the first step, the temperature is lowered, and the reaction of the second step is allowed. The reaction of the first step is usually allowed at a temperature of 40° C. or higher, 45° C. or higher, or 50° C. or higher, as for the lowest temperature, and usually 70° C. or lower, 65° C. or lower, or 60° C. or lower, as for the highest temperature. The reaction of the second step is usually allowed at a temperature of 5° C. or higher, 20° C. or higher, or30° C. or higher, as for the lowest temperature, and usually 60° C. or lower, 50° C. or lower, or 45° C. or lower, as for the highest temperature.

According to the present invention, in the reaction of the first step in the two-step reaction, culture obtained by the aforementioned culture method for algae may be used as it is, or may be used after concentration as described above. For example, the culture may be once centrifuged, and precipitated alga bodies may be used as a reaction product.

Further, before the reaction of the first step, pH of the reaction mixture may be adjusted to a weakly acidic or weakly alkaline pH.

The weakly acidic pH can be 3.0 to 6.5, or 4.0 to 6.0. The weakly alkaline pH can be 7.5 to 12.0, or 9.0 to 11.0.

As for the method of adding an alcohol before the reaction of the second step, an alcohol may be added to the reaction mixture obtained after the reaction of the first step, or after the liquid phase of the reaction mixture obtained after the reaction of the first step is removed by centrifugation or the like, an alcohol may be added to the reaction mixture for the reaction of the second step.

The alcohol can be added before the reaction of the second step at a concentration of, i.e., the reaction of the second step is performed at an alcohol concentration of, at least 5% or higher, 10% or higher, or 20% or higher. Further, the concentration is usually 70% or lower, 60% or lower, or50% or lower, as for the highest concentration.

As the alcohol to be added, there may be used a lower alcohol having a carbon number of 5 or smaller such as methanol, ethanol, propanol, isopropanol, butanol, pentanol, and ethylene glycol, or a higher alcohol having a carbon number of 6 or larger such as hexanol, heptanol, octanol, nonanol, decanol, undecanol, dodecanol, tridecanol, and tetradecanol.

The reaction of the first step (reaction at a mid-temperature) can be allowed for at least 5 minutes or longer, 10 minutes or longer, or 20 minutes or longer. The reaction of the first step can be allowed for usually 120 minutes or shorter, or 60 minutes or shorter. The reaction of the second step (reaction at a sub-mid-temperature) can be allowed for at least 10 minutes or longer, 30 minutes or longer, or 120 minutes or longer, as for the shortest reaction time, and can be allowed for usually 15 hours or shorter, 10 hours or shorter, or 5 hours or shorter, as for the longest reaction time.

As the method for collecting the fatty acid ester from the reaction product obtained after the two-step reaction, common methods for extracting fats and oils from algae can be used, and examples include, for example, treatment with organic solvent, ultrasonication, beads mill treatment, acid treatment, alkali treatment, enzyme treatment, hydrothermal treatment, supercritical treatment, microwave treatment, electromagnetic field treatment, compression treatment, and so forth. A method of eluting the fatty acid ester out of the cells, and collecting the fatty acid ester from the effluent can be employed.

Examples of the solvent that can be used for the organic solvent treatment performed after the two-step reaction include methanol, ethanol, 2-propanol, acetone, butanol, pentanol, hexanol, heptanol, octanol, chloroform, methyl acetate, ethyl acetate, dimethyl ether, diethyl ether, hexane, and so forth.

After the two-step reaction, the reaction mixture can be separated into a precipitate and supernatant by centrifugation. Further, after the two-step reaction, an organic solvent may be added, and the reaction mixture may be subjected to double-layer extraction with an aqueous layer and an organic solvent layer.

It is considered that the reason why addition of a catalyst is not required is that lipases in cells of microalga are made to be in a state that they can more easily act on lipids by the reaction of the first step, and these lipases catalyze transesterification of fats, oils, ceramides, phospholipids, phospholipids, and glycolipids with the alcohol added from the outside.

The transesterification catalyzed by a lipase is generally promoted by addition of an organic solvent other than alcohols. Therefore, in the reaction of the second step, an organic solvent may be added in an amount effective for promoting the reaction. Examples of such an organic solvent include, for example, hexane, heptane, isooctane, chloroform, ethyl acetate, petroleum ether, and so forth.

### EXAMPLES

Hereafter, the present invention will be explained more specifically with reference to the following non-limiting examples. In the examples, the Chlorella kessleri 11H strain (UTEX 263), and the Scenedesmus abundans UTEX 1358 strain obtained from the University of Texas at Austin, The Culture Collection of Algae (UTEX) (1 University Station A6700, Austin, Tex. 78712-0183, USA) were used.

### Example 1

### Culture of Microalga Chlorella kessleri 11H Strain

The Chlorella kessleri 11H strain was cultured at 30° C. and a light intensity of 7,000 luxes (culture apparatus: CL-301, TOMY) for 7 days in 800 mL of the 0.2× Gamborg's B5 medium (NIHON PHARMACEUTICAL) contained in a 1000 mL-volume medium bottle with blowing 400 mL/minute of a mixed gas of air and 3% CO₂, and the resultant culture was used as a preculture fluid. As the light source, white light from a fluorescent lamp was used. The preculture fluid in a volume of 16 mL was added to 800 mL of the 0.2× Gamborg's B5 medium contained in a 1000 mL-volume medium bottle, and culture was performed at a culture temperature of 30° C. and a light intensity of 7,000 luxes for 14 days with blowing 400 mL/minute of a mixed gas of air and 3% CO₂ into the medium.

0.2× Gamborg's B5 medium:

| | | |
|---|---|---|
| KNO₃ | 500 | mg/L |
| MgSO₄•7H₂O | 50 | mg/L |
| NaH₂PO₄•H₂O | 30 | mg/L |
| CaCl₂•2H₂O | 30 | mg/L |
| (NH₄)₂SO₄ | 26.8 | mg/L |
| Na₂-EDTA | 7.46 | mg/L |
| FeSO₄•7H₂O | 5.56 | mg/L |
| MnSO₄•H₂O | 2 | mg/L |
| H₃BO₃ | 0.6 | mg/L |
| ZnSO₄•7H₂O | 0.4 | mg/L |
| KI | 0.15 | mg/L |
| Na₂MoO₂•2H₂O | 0.05 | mg/L |
| CuSO₄•5H₂O | 0.005 | mg/L |
| CoCl₂•6H₂O | 0.005 | mg/L |

The medium was sterilized by autoclaving at 120° C. for 15 minutes.

### Example 2

### Examination of Temperature Condition for Reaction of First Step in Two-Step Reaction of Alga

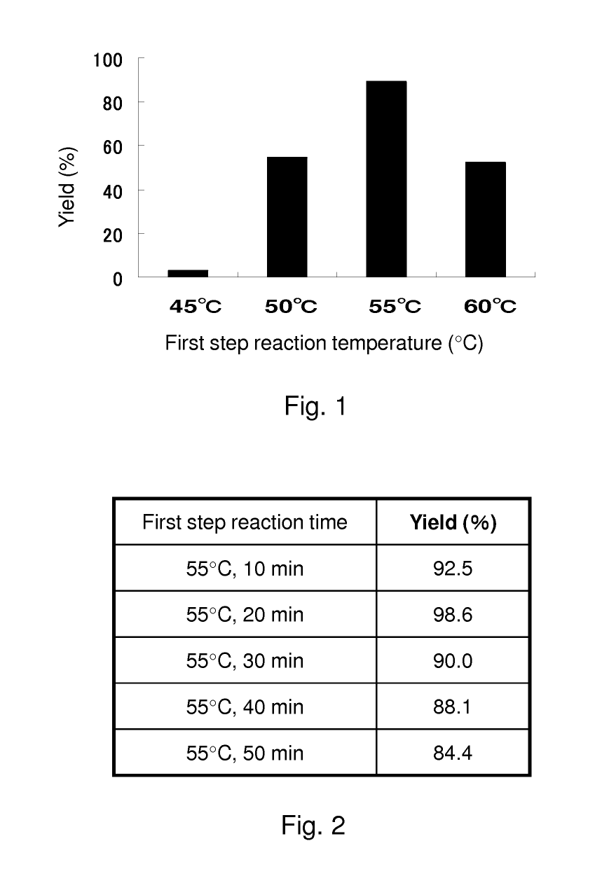

The culture fluid obtained in Example 1 was centrifuged, and sterilized water was added to the obtained precipitates to prepare a 1× suspension. The suspension was adjusted to pH 4.5 with a 1 N HCl solution, put into 1.5 ml-volume Eppendorf tubes in a volume of 1 ml each, and preincubated at temperatures of 45° C., 50° C., 55° C. and 60° C. for 10 minutes in a standing state. Then, each sample was incubated at the same temperature and 1000 rpm for 30 minutes, and centrifuged, and 200 μl of a 10% methanol solution was added to the obtained precipitates. Each sample was incubated at 42° C. and 1000 rpm for 5 hours to allow transesterification between fats and oils and methanol. Lipids were extracted from the obtained sample, and fatty acid methyl esters were measured. The measurement results are shown in FIG. 1. When the induction was performed at 45° C. for 30 minutes, and then incubation was performed at 42° C. for 5 hours, production of fatty acid methyl esters was hardly confirmed. In contrast, when the induction was performed at 50° C. or higher for 30 minutes, and then incubation was performed at 42° C. for 5 hours, production of fatty acid methyl esters was confirmed, and the yield was maximized at the induction temperature of 55° C.

### Example 3

### Examination of Reaction Time for Reaction of First Step in Two-Step Reaction of Alga

The culture fluid obtained in Example 1 was centrifuged, and sterilized water was added to the obtained precipitates to prepare a 1× suspension. The suspension was adjusted to pH 4.5 with a 1 N HCl solution, put into 1.5 ml-volume Eppendorf tubes in a volume of 1 ml each, and preincubated at 55° C. for 10 minutes in a standing state. Then, each sample was incubated at 55° C. and 1000 rpm for 10, 20, 30, 40, 50, or 60 minutes, and centrifuged, and 200 μl of a 10% methanol solution was added to the obtained precipitates. Each sample was incubated at 42° C. and 1000 rpm for 5 hours to allow transesterification between fats and oils and methanol. Lipids were extracted from the obtained sample, and fatty acid methyl esters were measured. The measurement results are shown in FIG. 2. When the induction was performed at 55° C. for 20 minutes, the yield of fatty acid methyl ester production increased compared with that obtained with induction at 55° C. for 10 minutes. However, when the induction was performed at 55° C. for 30 minutes or longer, the yield tended to decrease as the induction time increased.

### Example 4

### Examination of pH for Reaction of First Step in Two-Step Reaction of Alga

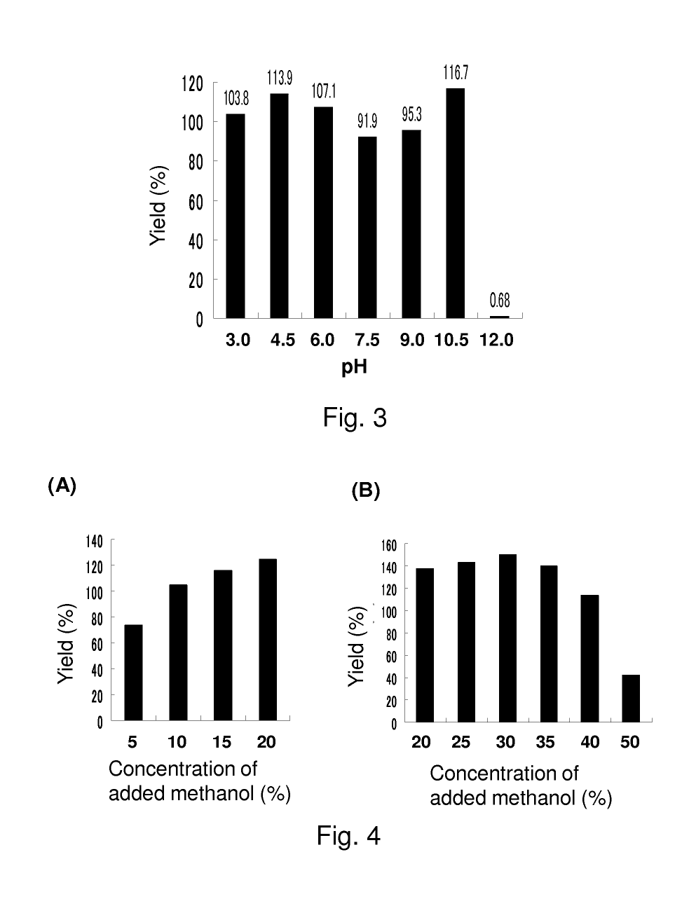

The culture fluid obtained in Example 1 was centrifuged, and sterilized water was added to the obtained precipitates to prepare a 1× suspension. The suspension was adjusted to various pH values with a 1 N HCl solution or a 1 N NaOH solution, put into 1.5 ml-volume Eppendorf tubes in a volume of 1 ml each, and preincubated at 55° C. for 5 minutes in a standing state. Then, each sample was incubated at 55° C. and 1000 rpm for 20 minutes, and centrifuged, and 200 μl of a 10% methanol solution was added to the obtained precipitates. Each sample was incubated at 42° C. and 1000 rpm for 5 hours to allow transesterification between fats and oils and methanol. Lipids were extracted from the obtained sample, and fatty acid methyl esters were measured. The measurement results are shown in FIG. 3. Production of fatty acid methyl esters was confirmed with pH in the range of 3.0 to 10.5, and within such a range, especially high yields were observed with two kinds of conditions, i.e., pH 4.5 in the weakly acidic region, and pH 10.5 in the weakly alkaline region.

### Example 5

### Examination of Addition Concentration of Alcohol for Two-Step Reaction of Alga

The culture fluid obtained in Example 1 was centrifuged, and sterilized water was added to the obtained precipitates to prepare a 1× suspension. The suspension was adjusted to pH 4.5 with a 1 N HCl solution, put into 1.5 ml-volume Eppendorf tubes in a volume of 1 ml each, and preincubated at 55° C. for 5 minutes in a standing state. Then, each sample was incubated at 55° C. and 1000 rpm for 20 minutes, and centrifuged, and 200 μl of a 5 to 50% methanol solution was added to the obtained precipitates. Each sample was incubated at 42° C. and 1000 rpm for 5 hours to allow transesterification between fats and oils and methanol. Lipids were extracted from the obtained sample, and fatty acid methyl esters were measured. The measurement results are shown in FIG. 4. With a concentration of added methanol up to 30%, the yield of the fatty acid methyl ester production increased with increase of the concentration. However, with a high methanol addition concentration of 35% or higher, the yield decreased with increase of the concentration.

### Example 6

### Examination of Reaction Time for Reaction of Second Step in Two-Step Reaction of Alga

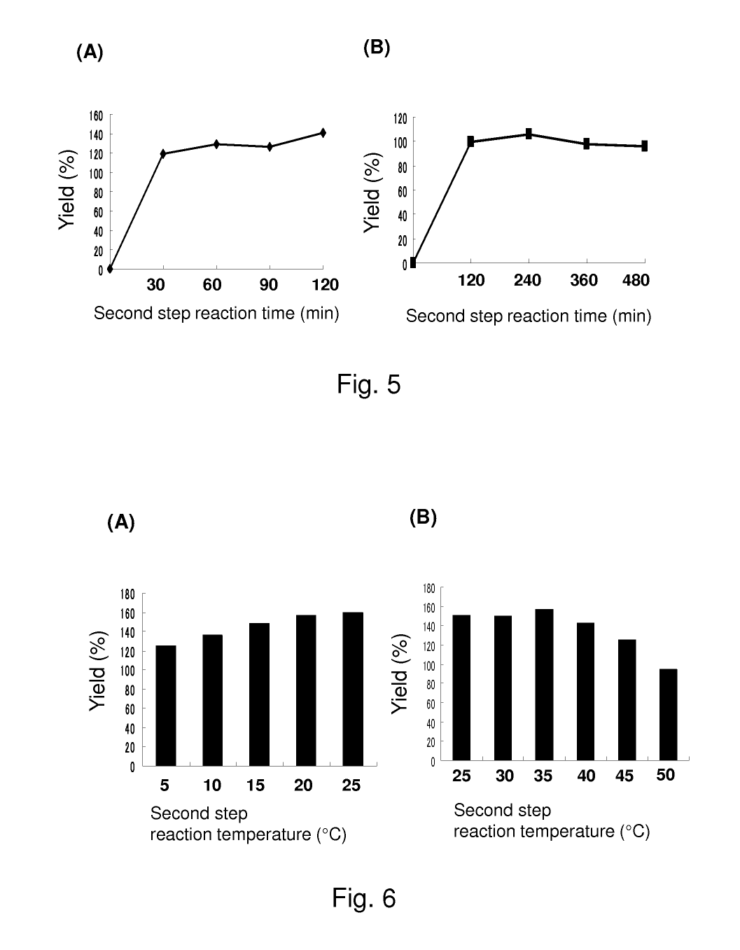

The culture fluid obtained in Example 1 was centrifuged, and sterilized water was added to the obtained precipitates to prepare a 1× suspension. The suspension was adjusted to pH 4.5 with a 1 N HCl solution, put into 1.5 ml-volume Eppendorf tubes in a volume of 1 ml each, and preincubated at 55° C. for 5 minutes in a standing state. Then, each sample was incubated at 55° C. and 1000 rpm for 20 minutes, and centrifuged, and 200 μl of a 30% methanol solution was added to the obtained precipitates. Each sample was incubated at 42° C. and 1000 rpm for various lengths of time to allow transesterification between fats and oils and methanol. Lipids were extracted from the obtained sample, and fatty acid methyl esters were measured. The measurement results are shown in FIG. 5. When the reaction time was lengthened from 30 minutes to 60, 90, 120, and 240 minutes, the yield of the fatty acid methyl ester production gradually increased as the reaction time became longer. However, with a reaction time of 360 minutes or longer, the yield tended to gradually decrease as the reaction time became longer.

### Example 7

### Examination of Temperature for Reaction of Second Step in Two-Step Reaction of Alga

The culture fluid obtained in Example 1 was centrifuged, and sterilized water was added to the obtained precipitates to prepare a 1× suspension. The suspension was adjusted to pH 4.5 with a 1 N HCl solution, put into 1.5 ml-volume Eppendorf tubes in a volume of 1 ml each, and preincubated at 55° C. for 5 minutes in a standing state. Then, each sample was incubated at 55° C. and 1000 rpm for 20 minutes, and centrifuged, and 200 μl of a 30% methanol solution was added to the obtained precipitates. The samples were incubated at various temperatures and 1000 rpm for 2 hours to allow transesterification between fats and oils and methanol. Lipids were extracted from the obtained samples, and fatty acid methyl esters were measured. The measurement results are shown in FIG. 6. Production of fatty acid methyl esters was confirmed even at a reaction temperature of 5° C., and the yield of the fatty acid esters increased with increase of the reaction temperature up to a reaction temperature of 35° C. However, at temperatures of 40° C. or higher, the yield tended to decrease with increase of the temperature.

### Example 8

### Examination of Type of Alcohol Added for Two-Step Reaction of Alga

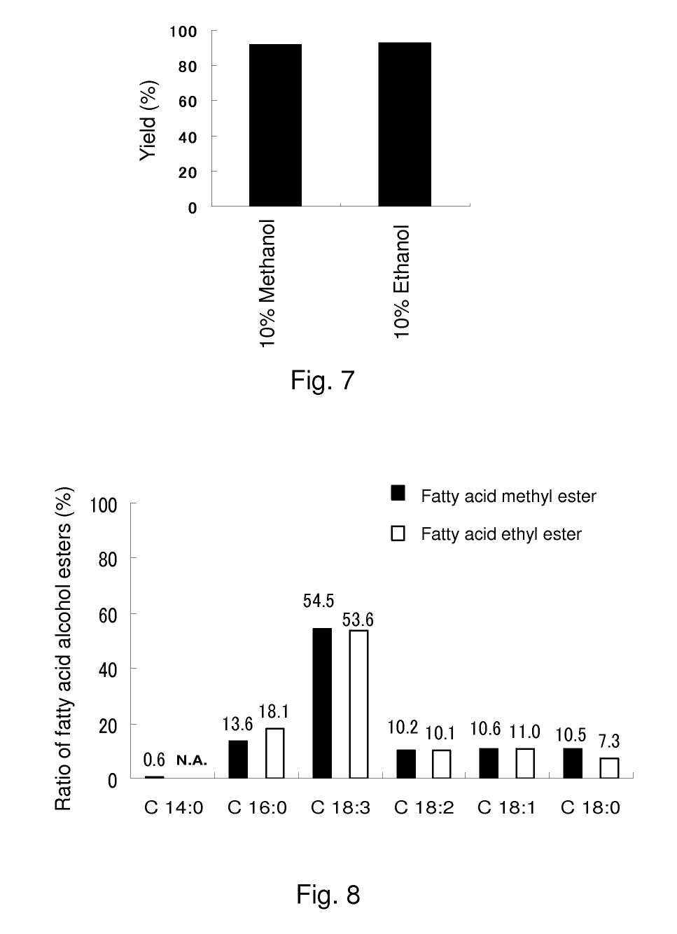

The culture fluid obtained in Example 1 was centrifuged, and sterilized water was added to the obtained precipitates to prepare a 1× suspension. The suspension was adjusted to pH 4.5 with a 1 N HCl solution, put into 1.5 ml-volume Eppendorf tubes in a volume of 1 ml each, and preincubated at 55° C. for 5 minutes in a standing state. Then, each sample was incubated at 55° C. and 1000 rpm for 20 minutes, and centrifuged, and 200 μl of a 10% methanol solution, a 10% ethanol solution or a 10% butanol solution was added to the obtained precipitates. Each sample was incubated at 42° C. and 1000 rpm for 5 hours to allow transesterification between fats and oils and methanol. Lipids were extracted from the obtained sample, and fatty acid methyl esters were measured. The measurement results are shown in FIG. 7. With addition of 10% ethanol, there was obtained a yield substantially comparable to that obtained with the addition of 10% methanol. Further, a plurality of bands of fatty acid butanol esters were confirmed with addition of butanol as in the cases where methanol or ethanol was added.

### Example 9

### Identification of Fatty Acid Alcohol Esters Produced by Two-Step Reaction

The culture fluid obtained in Example 1 was centrifuged, and sterilized water was added to the obtained precipitates to prepare a 1× suspension. The suspension was adjusted to pH 4.5 with a 1 N HCl solution, put into 1.5 ml-volume Eppendorf tubes in a volume of 1 ml each, and preincubated at 55° C. for 5 minutes in a standing state. Then, each sample was incubated at 55° C. and 1000 rpm for 20 minutes, and centrifuged, and 200 μl of a 10% methanol solution, or a 10% ethanol solution was added to the obtained precipitates. Each sample was incubated at 42° C. and 1000 rpm for 5 hours to allow transesterification between fats and oils and each alcohol. Lipids were extracted from the obtained sample, and fatty acid alcohol esters were identified. The results are shown in FIG. 8. The methanol addition group and the ethanol addition group showed substantially the same compositions of fatty acid alcohol esters. However, myristic acid ethyl ester was not analyzed in the case of addition of ethanol (indicated as N.A.). The α-linolenic acid alcohol ester content is the highest, and in addition to that ester, myristic acid methyl ester, palmitic acid alcohol esters, linolic acid alcohol esters, oleic acid alcoholic esters, and stearic acid alcohol esters were confirmed.

### Example 10

### Culture of Scenedesmus abundans UTEX 1358 Strain

The Scenedesmus abundans UTEX 1358 strain was cultured at 30° C. under an artificial sunshine condition at a light intensity of 7,000 luxes (gradient method using 11 hours each of bright period and dark period) in 100 mL of the Modified Bold 3N medium contained in a 500 mL-volume Erlenmeyer flask for 7 days with maintaining a CO₂ concentration of 1% in the incubator, and the resultant culture medium was used as a preculture fluid. As the light source, white light from a fluorescent lamp was used. The preculture fluid in a volume of 5 mL was added to 100 mL of the Modified Bold 3N medium contained in a 500 mL-volume Erlenmeyer flask, and culture was performed under the same conditions for 16 days.

### Modified Bold 3N Medium:

| | | |
|---|---|---|
| NaNO₃ | 750 | mg/L |
| MgSO₄•7H₂O | 75 | mg/L |
| KH₂PO₄ | 175 | mg/L |
| K₂HPO₄ | 75 | mg/L |
| CaCl₂•2H₂O | 25 | mg/L |
| NaCl | 25 | mg/L |
| Na₂EDTA•2H₂O | 4.5 | mg/L |
| FeCl₃•6H₂O | 0.582 | mg/L |
| MnCl₂•4H₂O | 0.246 | mg/L |
| ZnCl₂ | 0.03 | mg/L |
| CoCl₂•6H₂O | 0.012 | mg/L |
| Na₂MoO₄•2H₂O | 0.024 | mg/L |
| HEPES | 0.036 | mg/L |
| Thiamine | 1.1 | mg/L |
| Biotin | 0.025 | mg/L |
| Vitamin B₁₂ | 0.12 | mg/L |
| CaCO₃ | 0.2 | mg/L |
| Green house soil | 0.2 | tsp/L |

The medium was adjusted to pH 6.2 and then sterilized by autoclaving at 120° C. for 15 minutes.

### Example 11

### Two-Step Reaction with Scenedesmus abundans UTEX 1358 Strain

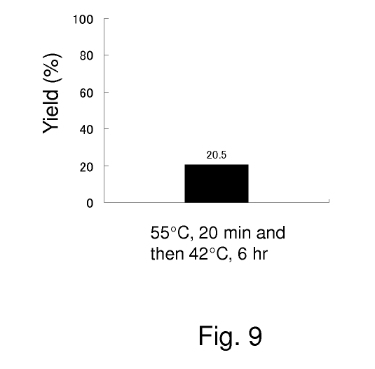

The culture fluid obtained in Example 10 in a volume of 100 mL was centrifuged, and sterilized water was added to the obtained precipitates to prepare a 1× suspension. The suspension was adjusted to pH 4.2 with a 1 N HCl solution, put into 1.5 ml-volume Eppendorf tubes in a volume of 1 ml, and preincubated at 55° C. for 5 minutes in a standing state. Then, the sample was incubated at 55° C. and 1000 rpm for 20 minutes, and centrifuged, and 200 μl of a 20% methanol solution was added to the obtained precipitates. The sample was incubated at 42° C. and 1000 rpm for 6 hours to allow transesterification between fats and oils and the alcohol. Lipids were extracted from the obtained sample, and fatty acid methyl esters were measured. The measurement result is shown in FIG. 9. Production of fatty acid methyl esters was also confirmed with the Scenedesmus abundans UTEX 1358 strain.

### INDUSTRIAL APPLICABILITY

According to the present invention, fatty acid esters can be efficiently produced.

While the invention has been described in detail with reference to preferred embodiments thereof, it will be apparent to one skilled in the art that various changes can be made and equivalents employed, without departing from the scope of the invention. Each of the aforementioned documents is incorporated by reference herein in its entirety.

1. A method for producing a fatty acid ester, which comprises:
(a) incubating a culture of a microalga at a mid-temperature,
(b) adding an alcohol to the culture, and reducing the temperature of the culture to a temperature lower than the mid-temperature, and
(c) collecting a fatty acid ester from an obtained reaction product.

2. The method according to claim 1, wherein said mid-temperature is 40° C. or higher.

3. The method according to claim 1, wherein said mid-temperature is 70° C. or lower.

4. The method according to claim 1, wherein said incubating of (a) is performed at a weakly acidic to weakly alkaline pH.

5. The method according to claim 1, wherein said temperature lower than the mid-temperature is 5° C. or higher.

6. The method according to claim 1, said temperature lower than the mid-temperature is 60° C. or lower.

7. The method according to claim 1, wherein said alcohol concentration is 5% or higher.

8. The method according to claim 1, wherein said alcohol concentration is 70% or lower.

9. The method according to claim 1, wherein said alcohol is a lower alcohol having a carbon number of 5 or smaller.

10. The method according to claim 1, wherein said alcohol is a higher alcohol having a carbon number of 6 or larger.

11. The method according to claim 1, wherein a product obtained after step (b) is treated with an organic solvent to extract a fatty acid ester, and the fatty acid ester is collected from an obtained extract.

12. The method according to claim 11, wherein a product obtained after step (b) is subjected to centrifugation, followed by treating the resulting precipitate with an organic solvent.

13. The method according to claim 11, wherein the organic solvent is selected from the group consisting of methanol, ethanol, 2-propanol, acetone, butanol, pentanol, hexanol, heptanol, octanol, chloroform, methyl acetate, ethyl acetate, dimethyl ether, diethyl ether, and hexane.

14. The method according to claim 1, wherein the microalga is an alga belonging to the phylum Chlorophyta.

15. The method according to claim 14, wherein the microalga is an alga belonging to the class Chlorophyceae, Trebouxiophyceae, or Prasinophyceae.

16. The method according to claim 15, wherein the microalga is an alga belonging to the class Chlorophyceae.
